(19) United States
(12) Patent Application Publication
(10) Pub. No.: US 2024/0212720 A1
(43) Pub. Date: Jun. 27, 2024

(54) Method and Apparatus for providing Timemarking based on Speech Recognition and Tag

(71) Applicants: Kyu Eun Lee, Seoul (KR); Hyoun-Joong Kong, Seoul (KR); Su-jin Kim, Seoul (KR); Hoorang Shin, Jeju-si (KR)

(72) Inventors: Kyu Eun Lee, Seoul (KR); Hyoun-Joong Kong, Seoul (KR); Su-jin Kim, Seoul (KR); Hoorang Shin, Jeju-si (KR)

(21) Appl. No.: 18/103,278

(22) Filed: Jan. 30, 2023

(30) Foreign Application Priority Data
Dec. 23, 2022 (KR) 10-2022-0183280

(51) Int. Cl. G11B 27/34; G06V 20/40; G10L 15/08; G10L 25/57

(52) U.S. Cl. CPC G11B 27/34; G06V 20/48; G06V 20/49; G10L 15/08; G10L 25/57; G06V 2201/03; G10L 2015/088

(57) ABSTRACT

Disclosed is a method and apparatus for providing timemarking based on speech recognition and a tag. The present embodiment provides a method and apparatus for providing timemarking based on speech recognition and a tag, in which after an audio and video of a selected medical video are separated, scene-based tag data extracted from the video and an audio-based text acquired from the audio are compared with a predefined keyword to determine a keyword for each section of the image, and then perform time marking for each section.

### BACKGROUND OF THE INVENTION

### 1. Field of the Invention

One embodiment of the present invention relates to a method and apparatus for providing timemarking based on speech recognition and a tag.

### 2. Description of the Related Art

The statements in this section merely provide background information related to the present embodiment and do not necessarily constitute related art.

Recently, in accordance with the spread of high-speed communication networks and the convergence of broadcasting and communication technologies, an internet protocol television (IPTV) or over the time (OTT) is coming up as an issue in the broadcasting industry. In the existing broadcasting, viewers may only passively watch contents transmitted as determined by the broadcasting station. However, in the IPTV or OTT, the viewers may watch the contents at the time they want, so that the IPTV or OTT is attracting attention as a service that may dramatically enhance the convenience and satisfaction of viewers.

A service that produces and provides various interactive broadcasting data using related metadata to a PC, a TV, a mobile terminal, etc., rather than simply broadcasting one video, is emerging. The metadata used for this service is largely divided into caption-based metadata having information about a video and content-based metadata having additional information for each segment or scene of the video.

The caption-based metadata is additional information about the entire video, such as title, director, characters, synopsis, genre, language, viewing grade, rating, and running time, and is widely used in related video recommendations, program guide services, and the like. In contrast, the content-based metadata is information representing a corresponding segment such as each segment (clip) consisting of a video, title, contents, type, and atmosphere of a scene, and appearance time and location on a screen of appearing objects (characters, props, places, background music, etc.), and may be used for highlights, program contents, service e-library, object-based advertisement, and commerce.

A service method is also being studied, in which a keyword that characterizes or represents a video scene is set for each scene to construct and provide metadata for the keyword. However, since a huge number of video contents are newly created and it takes too much time and cost to generate metadata for each video, it is difficult to extract a representative keyword based on the content for each video scene in reality.

Among conventional video scene search technologies, there is a technology for effectively searching for a desired scene in the video using a keyword related to video contents.

However, the conventional technologies have low accuracy of keywords (or tags) for each scene of the video or too many extracted keywords, so that it is difficult to determine which keyword is a keyword representing the scene, and it takes excessive effort and costs when the keyword is directly input because of non-automation of determination of keywords.

Therefore, in order to provide detailed information for each video scene, a more efficient method for dividing a video scene and extracting a keyword related to the divided video scene is necessarily required.

### SUMMARY OF THE INVENTION

### Technical Problem

An object of the present embodiment is to provide a method and apparatus for providing timemarking based on speech recognition and a tag, in which after an audio and video of a selected medical video are separated, scene-based tag data extracted from the video and an audio-based text acquired from the audio are compared with a predefined keyword to determine a keyword for each section of the image, and then perform timemarking for each section.

### Technical Solution

According to an aspect of the present embodiment, there is provided an apparatus for providing timemarking including: a keyword table that stores preset keywords for each type of surgery; a reference image selection unit that provides a reference image for each type of surgery; an image stream unit that receives a stream image for a specific surgery; a section keyword determination unit that determines an audio-based keyword based on the keywords stored in the keyword table and an audio of the stream image, determines a scene-based keyword based on the reference image and a video of the stream image, and determines a section keyword based on the audio-based keyword and the scene-based keyword matched to a specific section; and a time marking unit that time-matches the section keyword to the specific section.

### Advantageous Effects

According to the present embodiment as described above, the specific section corresponding to the predefined keyword is extracted from the medical video, timemarking is performed on the specific section, and the visual object corresponding to the keyword is matched to the time-marked section, such that the visual object can be output together with the medical video when the medical video is played.

### BRIEF DESCRIPTION OF THE DRAWINGS

FIG. 1 is a view schematically showing a timemarking system according to the present embodiment.

FIG. 2 is a view showing a timemarking apparatus according to the present embodiment.

FIG. 3 is a view describing a method for providing timemarking based on speech recognition and a tag according to the present embodiment.

FIG. 4 is a view showing a keyword table according to the present embodiment.

FIG. 5 is a view showing a selection of a text corresponding to an audio and an audio-based keyword matching with keywords stored in the keyword table according to the present embodiment.

FIG. 6 is a view showing control of each section of a stream image according to the present embodiment.

FIGS. 7a to 7d are views showing tags corresponding to scenes of a reference image according to the present embodiment.

FIG. 8 is a view showing a tag corresponding to a scene of the stream image according to the present embodiment.

FIG. 9 is a view showing a determination of a section keyword for each section in the stream image according to the present embodiment.

### DETAILED DESCRIPTION OF THE INVENTION

Hereinafter, the present embodiment will be described with reference to the accompanying drawings.

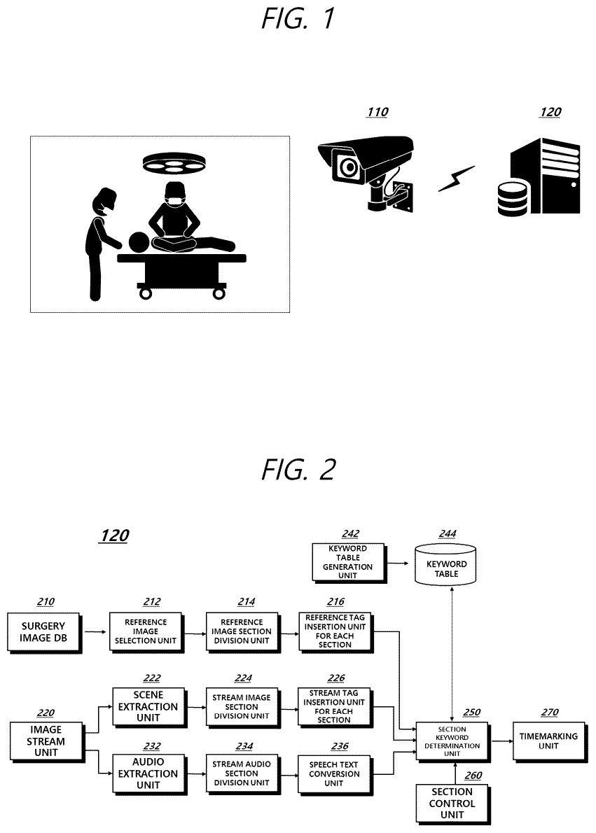

FIG. 1 is a view schematically showing a time marking system according to the present embodiment.

The timemarking system according to the present embodiment includes an image transmission apparatus 110 and a timemarking apparatus 120. Components of the timemarking system are not necessarily limited thereto.

The image transmission apparatus 110 is preferably installed in an operating room, but is not necessarily limited thereto. The image transmission apparatus 110 captures a surgery image in the operating room in real-time, and transmits the surgery image to an image stream unit 220, and simultaneously stores the surgery image in a surgery image DB 210. The image transmission apparatus 110 transmits the surgery image obtained by capturing a surgical procedure to the timemarking apparatus 120.

The timemarking apparatus 120 selects a reference image that meets a preset condition from a category the same as that of a stream image. The timemarking apparatus 120 selects an audio-based keyword that matches a text corresponding to an audio of the steam image and keywords stored in a keyword table 244. The timemarking apparatus 120 selects, as scene-based keywords, a tag corresponding to a scene of the stream image and a tag that matches a tag pre-stored in the reference image. The timemarking apparatus 120 maps the audio-based keyword and the scene-based keyword for each section of the stream image, and then applies a preset weight to determine a section keyword representing a section for each section.

The timemarking apparatus 120 time-marks the section keyword for each section. When the section keyword is selected, the timemarking apparatus 120 plays an image by moving the image to the corresponding section. For example, the timemarking apparatus 120 time-marks a surgery image with longer than 10 hours by determining a section keyword for each section with respect to the surgery image.

FIG. 2 is a view showing a timemarking apparatus according to the present embodiment.

The timemarking apparatus 120 according to the present embodiment includes a keyword table generation unit 242, the keyword table 244, a surgery image DB 210, a reference image selection unit 212, a reference image section division unit 214, a reference tag insertion unit 216 for each section, an image stream unit 220, a scene extraction unit 222, a stream image section division unit 224, a stream tag insertion unit 226 for each section, an audio extraction unit 232, a stream audio section division unit 234, a speech text conversion unit 236, a section keyword determination unit 250, a section control unit 260, and a timemarking unit 270. Components of the timemarking apparatus 120 are not necessarily limited thereto.

Each component of the timemarking apparatus 120 may be connected to a communication path for connecting software modules or hardware modules inside the apparatus to organically operate with each other. These components communicate with each other using one or more communication buses or signal lines.

Each component of the timemarking apparatus 120 shown in FIG. 2 refers to a unit for processing at least one function or operation, and may be implemented as a software module, a hardware module, and a combination of software and hardware.

The keyword table generation unit 242 generates a table in which keywords for each type of surgery are predefined and stores the predefined table in the keyword table. The keyword table generation unit 242 stores the plurality of image objects in an object DB 310 by matching a plurality of image objects for each keyword.

The keyword table 244 stores preset keywords for each type of surgery. The keyword table 244 stores a table in which a plurality of keywords are predefined for each type of surgery, and stores the plurality of image objects by being matched to each keyword.

The surgery image DB 210 records and stores the entire surgery image recorded by the image transmission apparatus 110. The surgery image DB 210 stores surgery information, surgery type, surgery name, surgeon, surgery method, and patient information by matching them to each surgery image. The surgery image DB 210 stores surgery images by being classified into brain surgery, cancer surgery, cancer surgery, surgical surgery, robotic surgery, etc. according to the surgery type. The surgery image DB 210 stores a plurality of surgery images.

The reference image selection unit 212 confirms surgical conditions (surgery name, surgeon, surgery method, and patient information) of the stream image, and selects a reference image from among the plurality of reference images when the reference image has a preset number or more of surgical conditions being matched.

The reference image selection unit 212 selects, as a reference image, any one image corresponding to a user command (surgery type, surgery name, surgeon, surgery method, and patient information) from among the plurality of surgery images. The reference image selection unit 212 selects an image, which may be used as a reference for other people, from among the plurality of surgery images stored in the surgery image DB 210 according to the user command. The reference image selection unit 212 selects a reference image corresponding to the input user command (patient age, patient gender, surgery method, surgery surgeon, and size of tumor in case of cancer surgery).

The reference image selection unit 212 provides a reference image for each type of surgery. The reference image selection unit 212 selects an image as a reference image when the image has a preset number or more of surgical conditions (surgery name, surgeon, surgery method, and patient information) of the plurality of surgeries by comparing the surgical conditions of the plurality of surgeries with surgery conditions of the stream image.

The reference image section division unit 214 divides the reference image into a plurality of sections. The reference image section division unit 214 divides the reference image into sections based on a preset unit time and a sequence number of each frame, or divides the reference image into each section by recognizing each scene of the reference image and grouping similar scenes using artificial intelligence.

The reference tag insertion unit 216 for each section inserts a tag corresponding to the reference image for each section of the reference image to generate a reference tag for each section.

The image stream unit 220 receives a stream image for a specific surgery. The image stream unit 220 outputs the surgery image received from the image transmission apparatus 110 in real-time using a display.

The scene extraction unit 222 separates only video data from the surgery image received from the image stream unit 220. The scene extraction unit 222 extracts a video from the stream image.

The stream image section division unit 224 divides the stream image into a plurality of sections. The stream image section division unit 224 divides the stream image into sections based on a preset unit time and a sequence number of each frame, or divides the stream image into each section by recognizing each scene of the stream image and grouping similar scenes using an artificial intelligence.

The stream tag insertion unit 226 for each section inserts a tag corresponding to a scene of the video for each section to generate a stream tag for each section.

The audio extraction unit 232 extracts an audio from the stream image.

The stream audio section division unit 234 divides the audio of the stream image into a plurality of sections. The stream audio section division unit 234 does not necessarily operate, but may selectively operate. The stream audio section division unit 234 divides the stream image into sections based on a preset unit time, or provides a section with similar contents by recognizing an audio-based text using an artificial intelligence.

The speech text conversion unit 236 separates only audio data from the surgery image received from the image stream unit 220. The speech text conversion unit 236 converts an audio into a text to generate an audio-based text.

The section keyword determination unit 250 determines an audio-based keyword based on the keywords stored in the keyword table 244 and an audio of the stream image. The section keyword determination unit 250 determines a scene-based keyword based on the reference image and a video of the stream image. The section keyword determination unit 250 determines a section keyword based on the audio-based keyword and scene-based keyword matched to a specific section.

The section keyword determination unit 250 determines an audio-based keyword based on the keywords stored in the keyword table and the audio-based text. The section keyword determination unit 250 determines a scene-based keyword based on the reference image and a stream tag for each section. The section keyword determination unit 250 determines a section keyword based on the audio-based keyword and scene-based keyword matched to a specific section.

The section keyword determination unit 250 confirms whether there is a keyword that matches the audio-based text among the keywords stored in the keyword table 244. When there are one or more keywords that match audio-based text data among the keywords stored in the keyword table 244, the section keyword determination unit 250 determines the keyword as an audio-based keyword.

The section keyword determination unit 250 compares the stream tag for each section with the reference tag for each section. The section keyword determination unit 250 determines, as a scene-based keyword, a tag that matches one of the stream tag for each section and the reference tag for each section.

The section keyword determination unit 250 matches the audio-based keyword and the scene-based keyword to each section. The section keyword determination unit 250 determines, as a section keyword, one of the audio-based keyword and the scene-based keyword by applying a weight to each of the audio-based keyword and the scene-based keyword matched to each section.

The section control unit 260 confirms a time when the audio-based keyword is selected from the stream image. The section control unit 260 resets, as a specific section, frames in which the image objects matched to the keywords stored in the keyword table exist from the time when the audio-based keyword is selected.

The timemarking unit 270 time-matches the section keyword to a specific section.

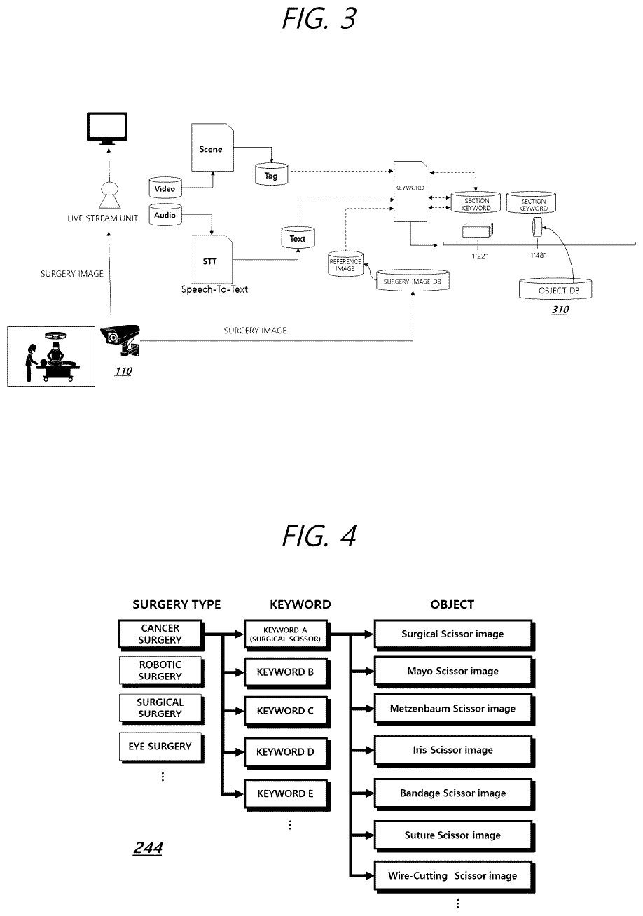

FIG. 3 is a view describing a method for providing timemarking based on speech recognition and a tag according to the present embodiment.

The image transmission apparatus 110 streams a medical video including a surgery name, a surgeon, a surgery method, and patient information.

The timemarking apparatus 120 extracts only a video from the medical video received from the image transmission apparatus 110 through streaming, and matches a tag for each section according to a specific scene in the image. The timemarking apparatus 120 extracts video data from the medical video. The timemarking apparatus 120 inserts a scene-based tag for the specific scene from the video data.

The timemarking apparatus 120 extracts only an audio from the medical video received from the image transmission apparatus 110 through streaming, and generates an audio-based text by converting an audio into a text. The timemarking apparatus 120 confirms whether there is a matching keyword by comparing a speech-based text with the predefined keyword table 244.

The timemarking apparatus 120 extracts a specific section existing in the keyword table 244 from the speech-based text. The timemarking apparatus 120 time-marks the specific section. When the specific section existing in the keyword table 244 does not exist in the speech-based text, the timemarking apparatus 120 may use the keywords by combining a text keyword with a visual object (e.g., surgical instrument). In other words, a surgical instrument, which may necessarily appear in an image when the text keyword appears, is stored in the form of an image, and a section in which the surgical instrument is used may be specified through video analysis.

The timemarking apparatus 120 generates an audio-based keyword by comparing the pre-stored keyword table 244 with the audio-based text. The timemarking apparatus 120 generates a scene-based keyword by comparing a tag matched to each section according to a specific scene with a tag matched to each section according to a specific scene of the reference image. The timemarking apparatus 120 determine a section keyword representing the section by applying a preset weight to the audio-based keyword and the scene-based keyword matched to the specific section.

The timemarking apparatus 120 extracts a tag corresponding to the specific keyword, a text, and a section corresponding to the reference image. The timemarking apparatus 120 time-marks the section keyword for each section. The timemarking apparatus 120 outputs the section keyword together with the video in a time-marked section when the video is played. The timemarking apparatus 120 outputs the visual object (e.g., surgical instrument) matched to the keywords together with the video in the time-marked section when the video is played.

The visual object (e.g., surgical instrument) is stored in the object DB 310 preset by a user. The timemarking apparatus 120 displays the visual object together with a time mark on an upper side of a playback player, only when the visual object matches the input keyword with a preset accuracy or higher.

The user may confirm a section keyword for each section using the visual object (e.g., surgical instrument). The user may grasp a progression state based on the visual object (e.g., surgical instrument). The user may receive an analysis service using the visual object (e.g., surgical instrument). When the user inputs (e.g., clicks) the visual object (e.g., surgical instrument), the timemarking apparatus 120 skips play of the image in the corresponding section.

The timemarking apparatus 120 extracts a specific section corresponding to the predefined keyword in the medical video (surgery video). The timemarking apparatus 120 performs timemarking for the specific section. The timemarking apparatus 120 plays a specific section corresponding to the keywords.

The timemarking apparatus 120 extracts a visual object (e.g., surgical instrument) specified in the medical video. The timemarking apparatus 120 extracts a tag defining a video or a specific scene in a media stream. The timemarking apparatus 120 plays keywords corresponding to the tags with timemarking data.

The timemarking apparatus 120 plays the keywords corresponding to the tags with the timemarking data, so that each scene may be efficiently recognized. The timemarking apparatus 120 outputs the keywords that match the tags to each scene without a separate indexing process, so that an end user may quickly recognize and find a specific scene in an image that the user wants to find.

The timemarking apparatus 120 extracts video and audio data consisting of continuous images from the video or media stream to define the data as a section keyword representing the section. The timemarking apparatus 120 extracts a plurality of keywords together with weights and priorities to determine a first section keyword having the highest priority. The timemarking apparatus 120 compares the keywords with the first section keyword based on metadata (title, description, presenter, affiliation, profile, tag, and pre-input) that are pre-input to the video or media stream to determine a second section keyword. The timemarking apparatus 120 selects a reference image similar to the pre-input metadata when the second section keyword does not exceed a validity score, and determines the second section keyword again by giving the score according to similarity obtained from a comparison of a reference section keyword presented and registered in the selected reference image with the first section keyword. When the second section keyword exceeds the validity score by repeating the above process, the timemarking apparatus 120 displays the second section keyword to the user and determines the final selection.

FIG. 4 is a view showing a keyword table according to the present embodiment.

The keyword table generation unit 242 generates a table in which keywords for each type of surgery are predefined and stores the predefined table in the keyword table. For example, the keyword table generation unit 242 predefines keywords a, b, c, d, and e for cancer surgery among surgeries. The keyword table generation unit 242 matches a plurality of image objects for each keyword and stores the plurality of image objects in the object DB 310.

For example, assuming that ‘keyword a’ is ‘surgical scissor’, the keyword table generation unit 242 matches a surgical scissor image, a mayo scissor image, a metzenbaum scissor image, an iris scissor image, a bandage scissor image, a suture scissor image, and a wire-cutting scissor image to ‘surgical scissor’ (keyword a), and stores the ‘keyword a’ in the object DB 310.

The keyword table generation unit 242 selects a plurality of keywords for each type of surgery (cancer surgery, surgical surgery, eye surgery, or robotic surgery) in advance. The keyword table generation unit 242 maps an image object for each of the plurality of keywords and stores the image object in the object DB 310.

FIG. 5 is a view showing a selection of a text corresponding to an audio and an audio-based keyword matching with keywords stored in the keyword table according to the present embodiment.

The section keyword determination unit 250 selects one or more of the keywords existing in the keyword table 244 from the audio-based text as an audio-based keyword. The section keyword determination unit 250 confirms whether there is a keyword that matches the audio-based text in the keyword table 244 previously generated by the keyword table generation unit 242.

When there are one or more keywords (e.g., Kocher's Point, 2% Lidocaine, and No. 20 Blade) matched with the audio-based text in the previously generated keyword table 244, the section keyword determination unit 250 selects the corresponding keyword as the audio-based keyword. For example, when there are two keywords matched with the audio-based text in the previously generated keyword table 244, the section keyword determination unit 250 selects the two keywords (e.g., Kocher's Point, 2% Lidocaine, and No. 20 Blade) as audio-based keywords.

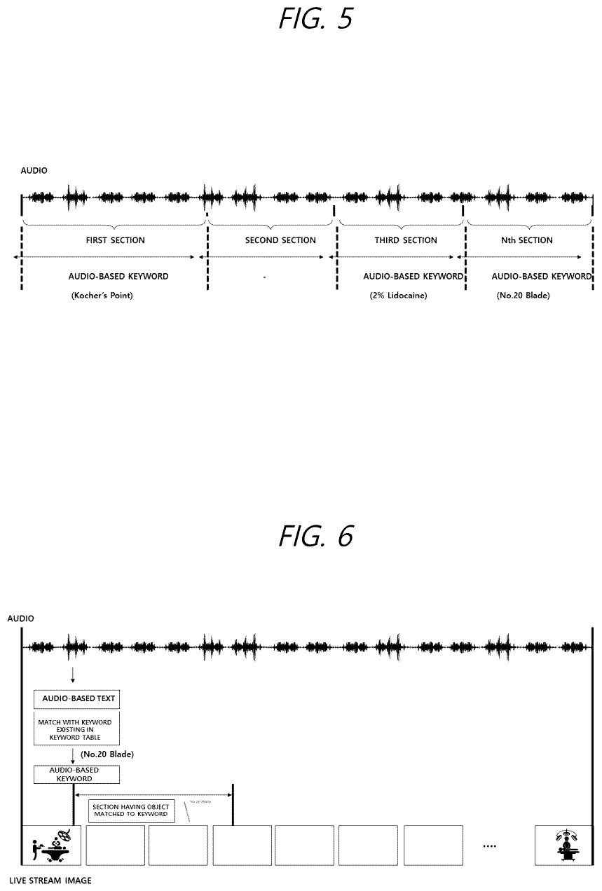

FIG. 6 is a view showing control of each section of a stream image according to the present embodiment.

When the audio-based text corresponding to the audio of the stream image matches the keywords existing in the keyword table 244, the section keyword determination unit 250 selects the keyword as an audio-based keyword (e.g., No. 20 Blade). The section control unit 260 confirms a time when the audio-based keyword (e.g., No. 20 Blade) is selected from the stream image. The section control unit 260 may set a specific section from the time when the audio-based keyword (e.g., No. 20 Blade) is selected to a frame having an object (e.g., No. 20 Blade image) matched to the keyword.

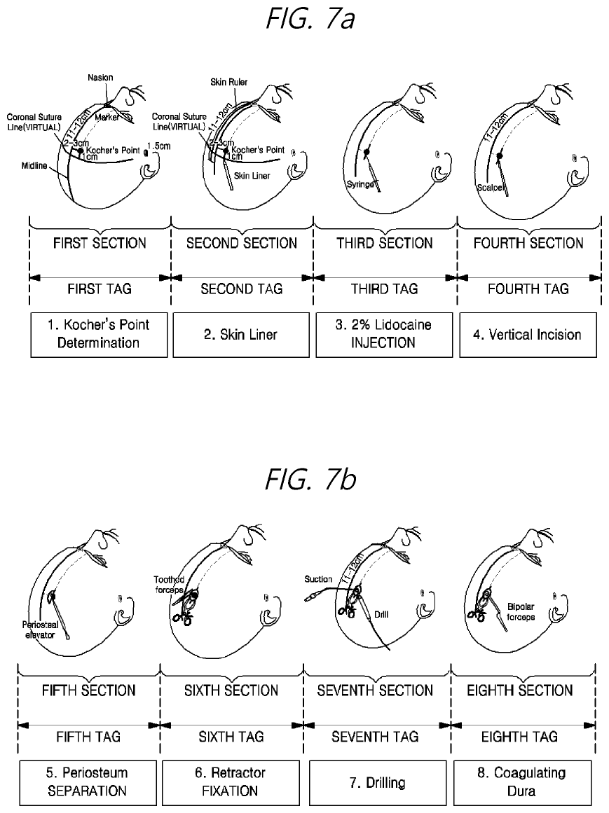

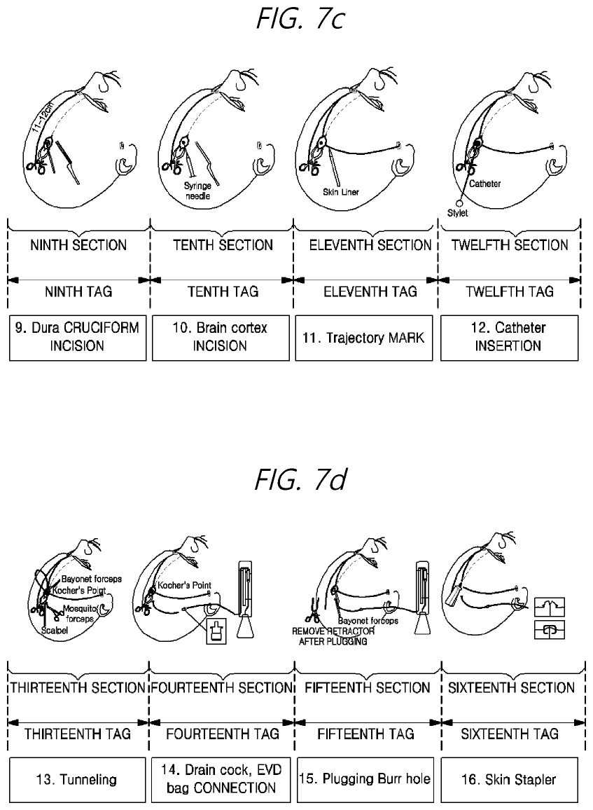

FIG. 7 is a view showing a tag corresponding to a scene of the reference image according to the present embodiment.

The reference image section division unit 214 divides the reference image into a plurality of sections. For example, the reference image section division unit 214 divides an external ventricular drainage (EVD) surgery image into first to sixteenth sections when the reference image relates to EVD surgery.

The reference image section division unit 214 provides the first section corresponding to attachment of a marker at a 1 cm point from Nasion and EAM anterior, in order to mark a guide of a position where a catheter is inserted in the EVD surgery image (reference image). Thereafter, the reference tag insertion unit 216 for each section inserts a first tag representing Kocher's point determination into the first section.

The reference image section division unit 214 provides the second section for a process of dressing after marking a vertical incision line in the EVD surgery image (reference image). Thereafter, the reference tag insertion unit 216 for each section inserts a second tag representing a skin liner into the second section.

The reference image section division unit 214 provides the third section for a process of injecting anesthesia into a lidocaine incision position and a subcutaneous tissue in the EVD surgical image (reference image). Thereafter, the reference tag insertion unit 216 for each section inserts a third tag representing 2% lidocaine injection into the third section.

The reference image section division unit 214 provides the fourth section for a vertical incision process using No. 20 Blade in the EVD surgery image (reference image). Thereafter, the reference tag insertion unit 216 for each section inserts a fourth tag representing vertical incision into the fourth section.

The reference image section division unit 214 provides the fifth section for a process of separating periosteal with a periosteal elevator in the EVD surgical image (reference image). Thereafter, the reference tag insertion unit 216 for each section inserts a fifth tag representing periosteum separation into the fifth section.

The reference image section division unit 214 provides the sixth section for a process of fixing an incision site using a toothed forceps and a retractor in the EVD surgery image (reference image). Thereafter, the reference tag insertion unit 216 for each section inserts a sixth tag representing retractor fixation into the sixth section.

The reference image section division unit 214 provides the seventh section for a drilling process in the EVD surgical image (reference image). Thereafter, the reference tag insertion unit 216 for each section inserts a seventh tag representing drilling into the seventh section.

The reference image section division unit 214 provides the eighth section for a process of coagulating a dura using a bipolar forceps in the EVD surgery image (reference image). Thereafter, the reference tag insertion unit 216 for each section inserts an eighth tag representing coagulating dura into the eighth section.

The reference image section division unit 214 provides the ninth section for a process of making incision of a dura into a cruciform (+) using No. 15 Blade and coagulating the incised dura using the bipolar forceps in the EVD surgery image (reference image). Thereafter, the reference tag insertion unit 216 for each section inserts a ninth tag representing dura cruciform incision into the ninth section.

The reference image section division unit 214 provides the tenth section for a process of making small incision of a brain cortex in a cruciform (+) using a syringe needle to form an opening through which the catheter may enter and coagulating a cortex opening site using the bipolar forceps, in the EVD surgery image (reference image). Thereafter, the reference tag insertion unit 216 for each section inserts a tenth tag representing a brain cortex into the tenth section.

The reference image section division unit 214 provides the eleventh section for a process of marking a trajectory in the EVD surgical image (reference image). Thereafter, the reference tag insertion unit 216 for each section inserts an eleventh tag representing a trajectory mark into the eleventh section.

The reference image section division unit 214 provides the twelfth section for a process of inserting the catheter at a depth of 5 cm, removing a stylet, and confirming whether a cerebrospinal fluid (CSF) is discharged, in the EVD surgery image (reference image). Thereafter, the reference tag insertion unit 216 for each section inserts a twelfth tag representing catheter insertion into the twelfth section.

The reference image section division unit 214 provides the thirteenth section for a process of finely incising the skin with No. 15 Blade to form an outlet to be tunneled to a subcutaneous layer by 4 cm to 5 cm, holding the catheter using a bayonet forceps, and taking a catheter distal part out of the skin using a mosquito forceps in the EVD surgery image (reference image). Thereafter, the reference tag insertion unit 216 for each section inserts a thirteenth tag representing tunneling into the thirteenth section.

The reference image section division unit 214 provides the fourteenth section for a process of connecting a drain cock and an EVD bag to a catheter distal end in the EVD surgery image (reference image). Thereafter, the reference tag insertion unit 216 for each section inserts a fourteenth tag representing EVD bag connection into the fourteenth section.

The reference image section division unit 214 provides the fifteenth section for a process of plugging a burr hole with a gelfoam having a size of 1.5 cm×1.5 cm in the EVD surgery image (reference image). Thereafter, the reference tag insertion unit 216 for each section inserts a fifteenth tag representing a plugging burr hole into the fifteenth section.

The reference image section division unit 214 provides the sixteenth section for a process of plugging a burr hole with a gelfoam having a size of 1.5 cm×1.5 cm in the EVD surgery image (reference image). Thereafter, the reference tag insertion unit 216 for each section inserts a sixteenth tag representing skin stapler into the sixteenth section.

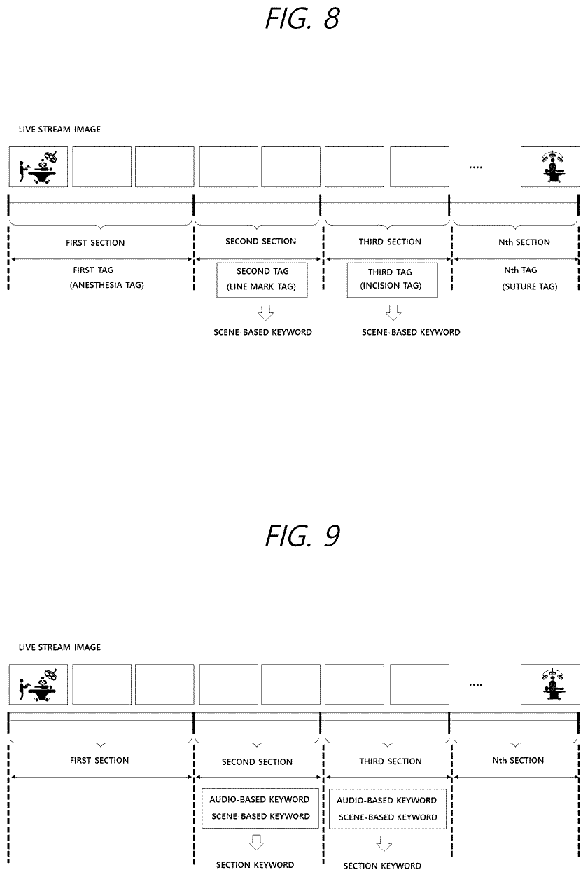

FIG. 8 is a view showing a tag corresponding to a scene of the stream image according to the present embodiment.

The stream image section division unit 224 divides the stream image into a plurality of sections. For example, the stream image section division unit 224 provides a first section corresponding to a process of injecting anesthesia into a surgical site when the steam image is the EVD surgery. Thereafter, the stream tag insertion unit 226 for each section inserts a first tag representing anesthesia into the first section.

The stream image section division unit 224 provides a second section corresponding to a mark of the vertical incision site in the stream image. Thereafter, the stream tag insertion unit 226 for each section inserts a second tag representing a line mark into the second section.

The stream image section division unit 224 provides a third section corresponding to a process of making incision of the vertical incision site in the stream image. Thereafter, the stream tag insertion unit 226 for each section inserts a third tag representing incision into the third section.

The stream image section division unit 224 provides an N^{th} section corresponding to suturing the surgical site in the stream image. Thereafter, the stream tag insertion unit 226 for each section inserts a N^{th} tag representing suture into the N^{th} section.

The section keyword determination unit 250 generates a comparison result by comparing the tags extracted from the stream tag insertion unit 226 for each section with the sections of the reference image. The section keyword determination unit 250 compares the tags of the reference image for each section with the tags of the stream image. The section keyword determination unit 250 selects a tag matched based on the comparison result as a scene-based keyword.

FIG. 9 is a view showing a determination of a section keyword for each section in the stream image according to the present embodiment.

The section keyword determination unit 250 matches the audio-based keyword and the scene-based keyword to each section. The section keyword determination unit 250 selects, as a section keyword, one of the audio-based keyword and the scene-based keyword by applying a weight to each of the audio-based keyword and the scene-based keyword matched to each section.

The timemarking unit 270 marks the section keyword for each section.

The reference image section division unit 214 may provide the sections based on a preset unit time and a sequence number of each frame, or provide the section by recognizing each scene and grouping similar scenes into a single section using artificial intelligence.

The section keyword determination unit 250 determines a word representing the section for each section of the stream image as a section keyword.

When the user selects the section keyword by performing timemarking of the section keyword for each section, the timemarking unit 270 moves the image to the corresponding section. The timemarking unit 270 moves the image to the corresponding section because the section keyword is matched to each specific section of the stream image.

The above description is merely illustrative of the technical idea of the present embodiment, and those skilled in the art to which this embodiment belongs will appreciate that various modifications and variations are possible without departing from the essential characteristics of the embodiments. Therefore, the present embodiments are not intended to limit the technical idea of the present disclosure, and the scope of the technical idea of the present disclosure is not limited by these embodiments. Accordingly, the scope of protection sought for by the present invention should be interpreted by the claims below, and all technical ideas within the scope equivalent thereto should be interpreted as being included in the scope of rights of the present invention.

1. An apparatus for providing timemarking, the apparatus comprising:
a keyword table that stores preset keywords for each type of surgery;
a reference image selection unit that provides a reference image for each type of surgery;
an image stream unit that receives a stream image for a specific surgery;
a section keyword determination unit that determines an audio-based keyword based on the keywords stored in the keyword table and an audio of the stream image, determines a scene-based keyword based on the reference image and a video of the stream image, and determines a section keyword based on the audio-based keyword and the scene-based keyword matched to a specific section; and
a time marking unit that time-matches the section keyword to the specific section.

2. The apparatus of claim 1, further comprising:
an audio extraction unit that extracts an audio from the stream image;
a speech text conversion unit that converts the audio into a text to generate an audio-based text;
a scene extraction unit that extracts a video from the stream image;
a stream image section division unit that divides the stream image into a plurality sections; and
a tag insertion unit for each section that inserts a tag corresponding to a scene of the video for each section to generate a stream tag for each section,
wherein the section keyword determination unit determines an audio-based keyword based on the keywords stored in the keyword table and the audio-based text, determines a scene-based keyword based on the reference image and a stream tag for each section, and determines a section keyword based on the audio-based keyword and the scene-based keyword matched to a specific section.

3. The apparatus of claim 1, wherein the keyword table stores a table in which a plurality of keywords are predefined for each type of surgery, and stores a plurality of image objects by matching the plurality of image objects to each keyword.

4. The apparatus of claim 1, further comprising:
a surgery image DB that stores a plurality of surgery images; and
a reference image selection unit that selects an image as a reference image when the image has a preset number or more of surgical conditions being matched by comparing surgical conditions of a plurality of surgeries with surgical conditions of the stream image.

5. The apparatus of claim 4, further comprising:
a reference image section division unit that divides the reference image into a plurality sections; and
a reference tag insertion unit for each section that inserts a tag corresponding to a scene of the reference image for each section of the reference image to generate a reference tag for each section.

6. The apparatus of claim 5, wherein the reference image section division unit divides the reference image into a plurality of sections based on a preset unit time and a sequence number of each frame, or divides the reference image into each section by recognizing each scene of the reference image and grouping similar scenes using an artificial intelligence.
